SEQUENCE LISTING (19 sequences)

SEQ ID NO: 1 - protein, 18 aa, artificial
  Consensus sequence FC-epitope HBsAg Loop 1 mAb5 and/or 6
  MISC_FEATURE (3)..(3): X = K or R
  MISC_FEATURE (6)..(6): X = T or M
  MISC_FEATURE (7)..(7): X = T or I
  MISC_FEATURE (8)..(8): X = P, T or L
  MISC_FEATURE (9)..(9): X = A or V
  MISC_FEATURE (12)..(12): X = N or T
  MISC_FEATURE (15)..(15): X = F or Y
PCXTCXXXXQGXSMXPSC

SEQ ID NO: 2 - protein, 18 aa, artificial
  FC epitope on HBsAg Loop 1 mAb 5 and/or 6
PCKTCTTPAQGNSMFPSC

SEQ ID NO: 3 - protein, 18 aa, artificial
  FC epitope on HBsAg Loop 1 mAb 5 and/or 6
PCRTCTTPAQGNSMFPSC

SEQ ID NO: 4 - protein, 18 aa, artificial
  FC epitope on HBsAg Loop 1 mAb 5 and/or 6
PCKTCTTPAQGTSMFPSC

SEQ ID NO: 5 - protein, 18 aa, artificial
  FC epitope on HBsAg Loop 1 mAb 5 and/or 6
PCRTCTTPAQGTSMFPSC

SEQ ID NO: 6 - protein, 18 aa, artificial
  FC epitope on HBsAg Loop 1 mAb 5 and/or 6
PCKTCTIPAQGTSMFPSC

SEQ ID NO: 7 - protein, 18 aa, artificial
  FC epitope on HBsAg Loop 1 mAb 5 and/or 6
PCRTCTIPAQGTSMFPSC

SEQ ID NO: 8 - protein, 18 aa, artificial
  FC epitope on HBsAg Loop 1 mAb 5 and/or 6
PCRTCTITAQGTSMFPSC

SEQ ID NO: 9 - protein, 18 aa, artificial
  FC epitope on HBsAg Loop 1 mAb 5 and/or 6
PCRTCTTPAQGTSMYPSC

SEQ ID NO: 10 - protein, 18 aa, artificial
  FC epitope on HBsAg Loop 1 mAb 5 and/or 6
PCRTCMTTAQGTSMYPSC

SEQ ID NO: 11 - protein, 18 aa, artificial
  FC epitope on HBsAg Loop 1 mAb 5 and/or 6
PCRTCMTTVQGTSMYPSC

SEQ ID NO: 12 - protein, 18 aa, artificial
  FC epitope on HBsAg Loop 1 mAb 5 and/or 6
PCRTCTTLAQGTSMFPSC

SEQ ID NO: 13 - protein, 18 aa, artificial
  FC epitope on HBsAg Loop 1 mAb 5 and/or 6
PCKTCTTLAQGTSMFPSC

SEQ ID NO: 14 - protein, 18 aa, artificial
  FC epitope on HBsAg Loop 1 mAb 5 and/or 6
PCKTCTTPAQGNSMYPSC

SEQ ID NO: 15 - protein, 13 aa, artificial
  Consensus sequence FC-epitope HBsAg Loop 2 mAb7 and/or 8
  MISC_FEATURE (4)..(4): X = T or S
  MISC_FEATURE (7)..(7): X = T or S
CCCXKPXDGNCTC

SEQ ID NO: 16 - protein, 13 aa, artificial
  FC epitope on HBsAg Loop 2 mAb 7 and/or 8
CCCTKPTDGNCTC

SEQ ID NO: 17 - protein, 13 aa, artificial
  FC epitope on HBsAg Loop 2 mAb 7 and/or 8
CCCTKPSDGNCTC

SEQ ID NO: 18 - protein, 13 aa, artificial
  FC epitope on HBsAg Loop 2 mAb 7 and/or 8
CCCSKPTDGNCTC

SEQ ID NO: 19 - protein, 13 aa, artificial
  FC epitope on HBsAg Loop 2 mAb 7 and/or 8
CCCSKPSDGNCTC

(19) United States
(12) Patent Application Publication
(10) Pub. No.: US 2019/0339275 A1
(43) Pub. Date: Nov. 7, 2019

(54) ASSAY FOR DETERMINING HEPATITIS B CLEARANCE

(71) Applicant: MELBOURNE HEALTH, Parkville, Victoria (AU)

(72) Inventors: Stephen Alister LOCARNINI, Melbourne (AU); Renae Brook WALSH, Melbourne (AU)

(21) Appl. No.: 16/348,837

(22) PCT Filed: Nov. 10, 2017
(86) PCT No.: PCT/IB2017/057055

§ 371 Date: May 9, 2019

Related U.S. Application Data
(60) Provisional application No. 62/420,760, filed on Nov. 11, 2016.

(51) Int. Cl. G01N 33/576

(52) U.S. Cl. CPC G01N 33/5764; G01N 2469/10; G01N 2469/20; G01N 33/5761

(57) ABSTRACT

The present invention relates to the identification of a profile of antibodies in an individual with chronic hepatitis B (CHB) wherein the existence of this profile is indicative that the individual will achieve or has achieved a functional cure (FC). The present invention further identifies an epitope profile or profile on Hepatitis B virus surface antigen (HBsAg) which represents targets for antibodies which enable a level of clearance to be achieved to reach a functional cure for CHB. Level of occupancy of the epitope profile is indicative that a functional cure will or will not be achieved.

This application claims the benefit of an priority to U.S. Provisional Patent Application No. 62/420,760, filed Nov. 11, 2016, the contents of which are incorporated herein by reference in their entirety. This specification refers to a Sequence Listing. The “ST25.txt” file is in ANSI format. The file is hereby incorporated in its entirety by reference from U.S. 62/420,760 into the subject specification.

### FIELD OF TECHNOLOGY

The present invention relates to the identification of a profile of antibodies in an individual with chronic hepatitis B (CHB) wherein the existence of this profile is indicative that the individual will achieve or has achieved a functional cure (FC). The present invention further identifies an epitope profile on Hepatitis B virus surface antigen (HBsAg) which represents targets for antibodies which enable a level of clearance to be achieved to reach a functional cure for CHB. Level of occupancy of the epitope profile is indicative that a functional cure will or will not be achieved.

### BACKGROUND

Bibliographic details of the publications referred to by author in this specification are collected alphabetically at the end of the description.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

Hepatitis B virus (HBV) is the causative agent of chronic hepatitis B (CHB). HBV affects liver function and can result in acute and chronic illness including cirrhosis and hepatic cell carcinoma. CHB can lead to liver failure and ultimately to death or serious impairment.

CHB can be managed using a modified interferon, pegylated interferon-alpha (pegIFN-α), or one of a number of nucleoside or nucleotide analogs that inhibit HBV DNA polymerase (Block et al. (2013) Antiviral Res. 98(1):27-34).

Furthermore, another form of treatment is the administration of HBV-specific immunoglobulins. However, obtaining highly specific and curative immunoglobulins from human plasma is difficult resulting in questionable benefit and high cost (Volz et al. (2015) International Meeting, Molecular Biology of Hepatitis B virus, Session IX: Antiviral therapy, 0-205, Germany).

These treatments alone cannot predict complete elimination of HBV. Part of the issue is defining an individual effectively in a state of cure. There is a need to define individuals which have or will achieve a functional cure. This is required to better and more effectively manage treatment of CHB. Importantly, if treatment can be halted or scaled back at time point earlier than might otherwise occur, this will result in significant economic advantage to patients and stakeholders in the healthcare industry. It also reduces the exposure of HBV to drugs and the selective pressure for development of resistance.

### SUMMARY

Amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A sequence listing is provided after the claims.

A summary of sequence identifiers used throughout the subject specification is provided in Table 1.

The present invention identifies a population of antibodies in subjects which results in clearance of Hepatitis B virus surface antigen (HBsAg) from serum and seroconversion to specific anti-HBsAg antibodies (anti-HBs). Such a virological and immunological profile is associated with a “functional cure” (FC) of chronic hepatitis B (CHB), as recently detailed by Lok et al. (2017) J. Hepatol. 67:847). The population of antibodies represents a clearance profile (CP) of antibodies (“CP-associated antibodies”) which induce or define a functional cure of CHB. The existence of HBsAg but with select epitopes occupied by the clearance profile of antibodies is indicative that a subject will reach a functional cure status. The absence of HB sAg but with the presence of the clearance profile of antibodies is indicative of the subject having achieved a functional cure. The presence of HBsAg but with select epitopes not occupied is indicative that the subject has not yet achieved a functional cure due to not having the requisite clearance profile (non-clearance profile or NCP) of antibodies. The identification of select epitopes on HBsAg and a clearance profile of antibodies enables treatment to be monitored and assessed, a state of functional cure to be determined following treatment or to predict patients who will progress to FC, and the ability to safely stop treatment.

The population of clearance profile antibodies binds to epitopes at Loop 1 and Loop 2 of HBsAg. This includes a single antibody that blocks a Loop 1 and Loop 2 epitope. These epitopes represent a clearance profile of epitopes. Availability of these epitopes means a state of non-functional cure as they are yet to be fully occupied by antibodies or other immune molecules such as a component of the complement pathway. Non-availability of the epitopes indicates they are all occupied by the antibodies in the subject and a state of functional cure will occur or has at least 80% probability of occurring. While for convenience, reference is made to the epitopes being occupied (or blocked) by an antibody, the present invention extends to other immune components or factors which may bind at or near the epitope(s).

In essence, the assay may detect circulating antibodies which have specificity for the HBsAg Loop 1 and Loop 2 epitopes or detect the epitopes to determine if they are occupied or not. Non-availability of an epitope indicates that either the antibody is bound there or another immune related molecule associated with an immune response. The assay is based in part on immobilized monoclonal antibodies directed to one of four epitopes located on Loop 1 and Loop 2 of HBsAg. Detection of an HBsAg means that the monoclonal antibody is directed to an epitope that is not occupied by an antibody or other immune molecule. When an assay is conducted and it is found that at least one epitope on Loop 1 and one epitope on Loop 2 are both occupied, then the indication is that a functional cure will or has occurred.

The assay of the present invention in essence comprises monoclonal antibodies (mAbs) immobilized directly or indirectly to solid discrete units, generally beads. The beads are optionally fluorescently labeled to enable inter alia flow cytometry or ease of identification. In a non-limiting embodiment, the beads are magnetic beads. Each mAb is specific for one of four epitopes on HBsAg. Where a mAb specific for a particular epitope is unable to bind to an HBsAg at the epitope, the epitope is said to be non-available for binding. This means the subject has an antibody bound to or close to that epitope. Both HBsAg or HBsAg complexed to an antibody may be assayed. Additionally, anti-HBs can be detected in a subject which has undergone a functional cure where no HBsAg will be circulating.

The subject assay enables a clinician to decide whether a subject on therapy will achieve a functional cure or has achieved a functional cure. The clinician can then cease treatment which is of significant economic saving to the healthcare industry, reduces HBV exposure to anti-virals which may result in the development of resistance and reduces the exposure of the subject to long term anti-viral therapy thereby minimizing potential adverse side effects.

Accordingly, taught herein is an assay to determine the likelihood that an individual with chronic hepatitis B (CHB) will achieve a functional cure (FC), the assay comprising determining a profile of the availability or non-availability of target epitopes on Hepatitis B virus surface antigen (HBsAg) for binding to anti-HBsAg antibodies (anti-HBs) wherein the target epitopes are located on Loop 1 of HBsAg defined by the consensus amino acid sequence:

(SEQ ID NO: 1)
PCX₁TCX₂X₃X₄X₅QGX₆SMX₇PSC

wherein:

### X₁ is K or R;

### X₂ is T or M;

### X₃ is T, or I;

### X₄ is P, T or L;

### X₅ is A or V;

### X₆ is N or T; and

### X₇ is F or Y,

and on Loop 2 of HBsAg defined by the consensus amino acid sequence:

(SEQ ID NO: 15)
CCC X₈KPX₉DGNCTC

wherein:
X₈ is T or S; and;

### X₉ is T or S

wherein the non-availability of at least one target epitope on each of Loop 1 and Loop 2 for binding to anti-HBsAg antibodies is indicative of those being occupied or blocked by anti-HBsAg antibodies or other molecules from the individual and wherein the individual is deemed likely to achieve a functional cure whereas availability of the target epitopes for binding to anti-HBsAg antibodies is indicative of the individual not having achieved a functional cure.

It is determined herein that the epitope on Loop 1 is defined by the consensus sequence set forth in SEQ ID NO: 1 or an amino acid sequence having at least 80% similarity thereto after optimal alignment. Examples include an epitope defined by an amino acid sequence selected from the list comprising SEQ ID NOs:2 through 14 or an amino acid sequence having at least 80% similarity to any one of SEQ ID NOs:2 through 14 after optimal alignment.

The epitope on Loop 2 is defined by the consensus sequence set forth in SEQ ID NO: 15 or an amino acid sequence having at least 80% similarity thereto after optimal alignment. Examples include an epitope defined by an amino acid sequence selected from the list comprising SEQ ID NOs:16 through 19 or an amino acid sequence having at least 80% similarity to any one of SEQ ID NOs: 16 through 19 after optimal alignment.

The present invention encompasses the binding of at least one epitope on Loop 1 (e.g. defined by binding of mAbs5 and 6) and at least one epitope on Loop 2 (e.g. defined by binding of mAbs7 and 8) or a single antibody which blocks the availability of the epitopes on Loop 1 and Loop 2. The mAbs 5, 6, 7, and 8 have been previously known and described at RFHBs 1, 2, 4 and 7 by Waters et al. (1986) J. Gen. Hep. 67:2467; Waters et al. (1991) Virus Res. 22:1) and Lever et al. (1988) Viral Hep. And Liver Dis.: 961). The individual being assayed may also have the epitopes occupied, not by an immunoglobulin, but by another molecule such as, for example, an innate immunity molecule. The present assay is defined as a “4-Plex” assay since up to four epitopes may be assayed defined by the consensus sequences of SEQ ID NOs: 1 and 15. Hence, the 4-Plex assay detects as a minimum at least one epitope at Loop 1 and at least one epitope at Loop 2, up to four epitopes. Accordingly, a “4-Plex” assay include the 2-Plex and 3-Plex assay.

A therapeutic protocol is enabled whereby a decision to cease treatment is made once a clearance profile of antibodies (CP-associated antibodies) is generated by the subject being managed and treated. Monitoring of treatment is also enabled herein as well as monitoring for individuals who naturally clear the virus.

Kits useful in performing the assay are encompassed by the present invention.

A list of abbreviations used throughout the subject specification is provided in Table 2.

TABLE 1
Summary of sequence identifiers

| SEQ ID NO | DESCRIPTION |
|---|---|
| 1 | [symbol] sensus sequence of FC-epitope on HBsAg Loop 1 |
| | which binds mAb5 and/or 6 |
| 2-14 | [symbol] ino acid sequence of FC epitope on HBsAg Loop 1 |
| | which binds mAb5 and/or 6 |
| 15 | [symbol] sensus sequence of FC-epitope on HBsAg Loop 2 |
| | which binds mAb7 and/or 8 |
| 16-19 | [symbol] ino acid sequence of FC epitope on HBsAg Loop 2 |
| | which binds mAb7 and/or 8 |

[symbol]

indicates data missing or illegible when filed

TABLE 2
Abbreviations

| ABBREVIATION | DESCRIPTION |
|---|---|
| ADV | Adefovir dipivoxil |
| Anti-HBs/Anti-HBsAg | Antibodies to HBV surface antigen |
| CHB | Chronic Hepatitis B |
| CP | Clearance profile |
| ETV | Entecavir |
| FC | Functional cure |
| HBsAg | HBV surface antigen |
| HBV | Hepatitis B virus |
| HCC | Hepatic Cellular Carcinoma |
| mAb | Monoclonal antibody |
| NCP | Non-clearance profile |
| “a” determinant | Ag antigenic region, residues 99-169 (as shown |
| | in FIG. 2) |
| HBsAg-AB/HBsAg-Ab | Complex of HBsAg with anti-HBs antibody |
| PE | Phycoerythrin |
| TDF | Tenofovir disoproxil fumarate |
| 4-Plex assay | [symbol] say which determines up to four epitopes |
| | wherein at least one |
| | [symbol] e is on Loop 1 of HBsAg and at least one |
| | epitope is on Loop 2 of HBsAg and includes a |
| | 2-Plex and 3-Plex assay |

[symbol]

indicates data missing or illegible when filed

In some aspects, the present disclosure provides an assay to determine the likelihood that an individual with chronic hepatitis B (CHB) will achieve a functional cure (FC), said assay comprising determining a profile of the availability or non-availability of target epitopes on Hepatitis B virus surface antigen (HBsAg) for binding to anti-HBsAg antibodies wherein the target epitopes are located on Loop 1 of HBsAg defined by the consensus amino acid sequence: PCX₁TCX₂X₃X₄X₅QGX₆SMX₇PSC (SEQ ID NO: 1) wherein: X₁ is K or R; X₂ is T or M; X₃ is T, or I; X₄ is P, T or L; X₅ is A or V; X₆ is N or T; and X₇ is F or Y, and on Loop 2 of HBsAg defined by the consensus amino acid sequence: CCC X₈KPX₉DGNCTC (SEQ ID NO:15) wherein: X₈ is T or S; and X₉ is T or S, wherein the non-availability of at least one target epitope on each of Loop 1 and Loop 2 for binding to anti-HBsAg antibodies is indicative of those being occupied or blocked by anti-HBsAg antibodies or other molecules from the individual and wherein the individual is deemed likely to achieve a FC whereas availability of the target epitopes for binding to anti-HB sAg antibodies is indicative of the individual not having achieved a FC.

In some embodiments, said assay comprises contacting a blood-derived sample comprising HBsAg from the individual with monoclonal antibodies (mAbs) specific for the target epitopes on HBsAg, said mAbs immobilized to a solid support, under conditions sufficient to capture HBsAg if the epitopes are not occupied by anti-HBsAg antibodies or other molecules generated by the individual.

In some embodiments, said assay comprises: (a) immobilizing to a support a set of mAbs, each mAb with a specificity to one of the target epitopes on HBsAg; (b) contacting the immobilized mAbs with a blood-derived sample from the individual under conditions sufficient to permit capturing of HBsAg if present; and (c) determining which immobilized mAbs have captured HBsAg and which have not to provide a profile of available and non-available epitopes on HBsAg.

In some embodiments, said assay comprises screening for antibodies in the individual which have the capacity to bind to or block the select epitopes on HBsAg wherein the presence of the antibodies in the absence of HBsAg is indicative of the individual having achieved a FC.

In some embodiments, the blood-derived sample is serum or a fraction of serum. In some embodiments, the serum is standardized for a level of HBsAg. In some embodiments, the individual is under treatment for CHB. In some embodiments, the treatment is a nucleotide or nucleoside analog, anti-viral, an interferon or anti-HBV antibodies.

In some embodiments, non-availability of binding at an epitope defined by SEQ ID NO:1 and SEQ ID NO:15 is indicative of a clearance profile (CP) of antibodies or other molecules associated with a FC and availability of binding an epitope defined by SEQ ID NO: 1 and SEQ ID NO: 15 is indicative of a non-clearance profile (NCP) of antibodies or other molecules and a FC has not be achieved.

In some embodiments, the epitope at Loop 1 of HBsAg is defined by an amino acid sequence selected from the group consisting of SEQ ID NOs:2 through 14. In some embodiments, the epitope at Loop 2 of HBsAg is defined by an amino acid sequence selected from the group consisting of SEQ ID NOs: 16 through 19. In some embodiments, the assay is a 2-Plex, 3-Plex or 4-Plex assay.

In some aspects, the present disclosure provides a method for determining when treatment of an individual for CHB can cease, the method comprising screening for the presence of a profile of antibodies or other molecules in the individual which constitutes antibodies or other molecules associated with a CP which are specific for target epitopes on HBsAg as determined by the assay of any one of the methods described herein, wherein the presence of the CP-associated antibodies or other molecules is indicative of a decision to cease treatment and the absence of CP-associated antibodies or other molecules is indicative of a decision not to cease treatment.

In some aspects, the present disclosure provides a method of managing treatment of CHB in an individual wherein the treatment comprises the administration of a nucleotide or nucleoside analog, an anti-viral, an interferon or anti-HBs, wherein the method comprises screening a sample from the individual for: (i) circulating antibodies or other molecules associated with a CP which the target epitopes on HBsAg; (ii) circulating HBsAg which have non-available epitopes for binding to this profile of antibodies or other molecules; or (iii) circulating HBsAg complexed to antibodies (HBsAg-Ab) or other molecules which have non-available epitopes for binding to this profile of antibodies or other molecules; wherein the target epitopes are determined by the assay of any one of the methods described herein, wherein if the profile of antibodies or other molecules exist in the absence of circulating HBsAg or if HBsAg exist but the epitopes not available for binding, then ceasing the anti-viral treatment can be recommended, wherein if the profile of antibodies or other molecules does not exist and circulating HBsAg is present then not ceasing the anti-viral treatment can be recommended.

### BRIEF DESCRIPTION OF THE FIGURES

FIG. 1 is a diagrammatic representation showing HBsAg epitope profile assay predictive biomarker of FC (assay 1) and anti-HBs development (assay 2). Assay 1: 1 Magnetic bead; 2. Capture Ab; 4-Plex mouse anti-HBs mAbs to HBsAg ‘a’ determinant; 3. Patient HBsAg sample; 4. Reporter Ab; PE conjugated polyclonal anti-HBs. Assay 2: 1 Magnetic bead; 2. Capture Ab: 2-Plex mouse anti-HBs mAbs to HBsAg ‘a’ determinant; 3. Patient sample: complexed anti-HBs (with HBsAg); 4. Reporter Ab: HRP conjugated goat anti-human IgG Fc domain.

FIG. 2 is a diagrammatic representation ofHBsAg epitope occupancy assay of the anti-HBs response associated with FC. Assay 3: 1. Magnetic bead; 2. Capture Ab: 4-plex mouse anti-HBs mABs to HBsAg “a” determinant; 3. Reference HBsAg sample. Ag only vs Ag pre-incubated with anti-HBs sample; 4. Reporter Ab: PE conjugated polyclonal anti-HBs.

FIG. 3 is a diagrammatic representation showing (A) CHB cure patients exhibition to HBsAg CP; and (B) non-cure/non-responder patient have an HBsAg NCP.

FIG. 4 is a graphical representation of HBsAg CP in CHB cure.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

As used in the subject specification, the singular forms “a”, “an” and “the” include plural aspects unless the context clearly dictates otherwise. Thus, for example, reference to “a profile” includes a single profile, as well as two or more profiles; reference to “an antibody” includes a single antibody, as well as two or more antibodies; reference to “the disclosure” includes a single and multiple aspects taught by the disclosure; and so forth. Aspects are enabled across the width of the invention. Aspects taught and enabled herein are encompassed by the term “invention”. Any variants and derivatives contemplated herein are encompassed by “forms” of the invention.

The present specification teaches a protocol to determine whether a subject with chronic hepatitis B (CHB) will achieve a functional cure or who has achieved a functional cure. In terms of a likelihood of achieving a functional cure, the protocol comprises:

(i) ascertaining the availability or non-availability of select epitopes (i.e. up to four CP-associated epitopes, using mAbs 5, 6, 7 and 8) on Hepatitis B virus (HBV) surface antigen (HBsAg);
(ii) determining whether the ascertained availability or non-availability of the select epitopes forms a profile of epitopes which bind to antibodies associated with a functional cure; wherein non-availability of this profile of epitopes is indicative of the epitopes being occupied by the subjects' antibodies which will result in a functional cure being achieved.

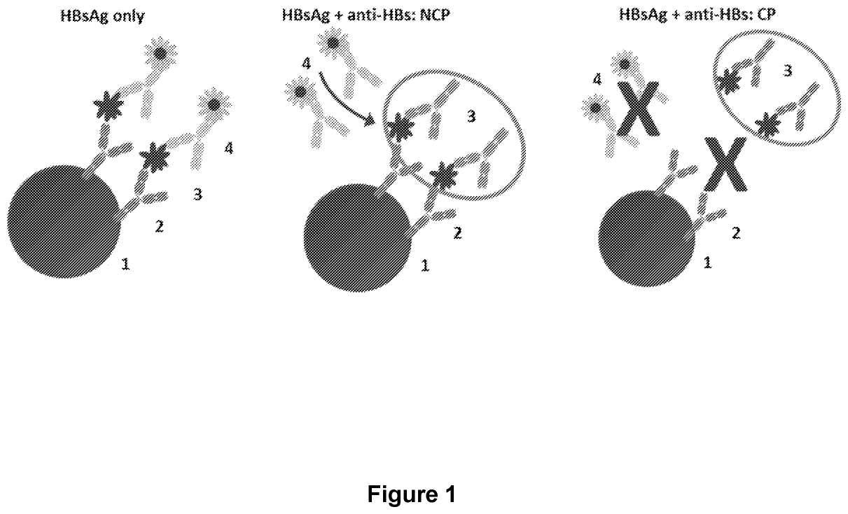

When a functional cure has been achieved, the key indicator is the presence of antibodies in the subject which are specific for that profile of HB sAg epitopes associated with a functional cure, in the absence of serum HBsAg. These antibodies induce a CP on a reference HBsAg sample, see FIG. 1. The protocol in this instance comprises ascertaining the presence of a population of antibodies specific for up to four epitopes on HBsAg by determining whether a population of antibodies in a blood-derived sample from a subject, if present, binds to a profile of epitopes on HBsAg, which epitopes bind to antibodies associated with a functional cure; wherein the presence of the antibodies to the profile of epitopes is indicative of the subject having achieved a functional cure. There are four epitopes, at least one on Loop 1 of HB sAg defined by SEQ ID NO:1 and at least one on Loop 2 of HBsAg defined by SEQ ID NO: 15. Hence, the assay includes a 2-Plex, 3-Plex and 4-Plex assay format.

Enabled herein is a protocol to determine whether a subject with chronic hepatitis B (CHB) will achieve a functional cure or who has achieved a functional cure, the protocol comprising determining a profile of the availability or non-availability of target epitopes on HBsAg or a population of antibodies in a sample from the subject which bind to these epitopes, wherein the profile of non-available epitopes on HBsAg is indicative of a likely progression to a functional cure and the presence of antibodies in the subject which are specific for the profile of epitopes is indicative of a functional cure having been achieved; wherein the target epitopes are those defined by the consensus sequence SEQ ID NO: 1 on Loop 1 of HBsAg and SEQ ID NO: 15 on Loop 2 of HBsAg. The latter feature is in the absence of detectable circulating HBsAg.

Reference to a sample includes a biological sample such as whole blood, serum, plasma or other body fluid which may contain circulating antibodies or HBsAg. Further enabled is an assay to determine the likelihood that an individual with CHB will achieve a functional cure, the assay comprising determining a profile of the availability or non-availability of target epitopes on Hepatitis B virus serface antigen (HBsAg) which bind to anti-HBsAg antibodies from the individual and induce clearance of HBsAg wherein the non-availability of the target epitopes is indicative of those being occupied by the anti-HBsAg antibodies wherein the individual is deemed likely to achieve a functional cure; wherein the target epitopes are those defined by the consensus sequence SEQ ID NO:1 on Loop 1 of HBsAg and SEQ ID NO:15 on Loop 2 of HBsAg.

The present protocol may also be referred to as an assay, method, procedure, a step or other like term such as a 2-Plex, 3-Plex or 4-Plex assay. Each of these terms may be used interchangeably and refers to a set of artificial actions which enables a physical transformation into a particular result. For the absence of doubt, up to four epitopes are assayed or circulating antibodies specific for up to four epitopes provided that at least one epitope is on Loop 1 and one epitope is on Loop 2, each as defined by SEQ ID NOs:1 and 15, respectively. This result is a determination whether a subject having been diagnosed with CHB will achieve a functional cure, which includes a likelihood of the functional curing being achieved or an already achieved functional cure. Generally, the subject is on-treatment such as with a nucleotide or nucleoside analog, anti-viral agent, an interferon or anti-HBV immunoglobulin. Hence, the subject method can identify such individuals. The present method is also useful for monitoring on-going treatment or those who have achieved a functional cure.

The term “functional cure” refers to a functional cure of CHB and is defined by loss of detectable HBsAg and seroconversion to detectable (or inducible) after exposure to anti-HBsAg antibodies (also referred to herein as “anti-HBs antibodies” or “anti-HBs”) which include a population of antibodies which selectively bind to select epitopes on HBsAg which when occupied by anti-HBs antibodies results in clearance of HBsAg and ultimately HBV wherein the target epitopes are those defined by the consensus sequence SEQ ID NO: 1 on Loop 1 of HBsAg and SEQ ID NO:15 on Loop 2 of HBsAg. Serum HBV virions, DNA or HbsAg are non-detectable in a fully cured subject.

For brevity, the antibodies within an anti-HBs response which result in functional cure are referred to as “clearance antibodies”. These antibodies define a “clearance profile” of antibodies or “CP-associated antibodies” which target the specific HBsAg epitopes and which ultimately result in clearance of HBsAg and the functional cure. The epitopes are referred to as “clearance epitopes” or “CP epitopes” meaning once occupied by antibodies in the host will result or likely result in a functional cure. Hence, a “clearance profile” can refer to the profile of non-available epitopes on HBsAg or the suite or population of an individual's antibodies which occupy these epitopes and when they do occupy the epitopes, a functional cure will be achieved. The clearance profile of antibodies or epitopes on HBsAg represent biomarkers of the potential or likelihood that a subject on treatment will achieve a functional cure, wherein the target epitopes are those defined by the consensus sequence SEQ ID NO: 1 on Loop 1 of HBsAg and SEQ ID NO: 15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs: 1 or 15. Reference to a “likelihood” of a functional cure generally means that the likelihood is 100%, that is, once a clearance profile of antibodies is detected, the subject will reach a state of functional cure or, in the absence of circulating HBsAg, has achieved a functional cure. However, as in any biological system, variability can occur. Hence, for the purposes of the present invention, reference to a “likelihood” of a functional cure means at least 80% probability that a subject with a clearance profile of antibodies will achieve a functional cure. By “at least 80% means 80, 81, 82, 83, 84, 85, 86, 87, 88, 89, 90, 91, 92, 93, 94, 95, 96, 97, 98, 99 or 100%.

The “clearance profile of epitopes” or “CP of epitopes” on HBsAg is the presence of available or non-available epitopes on HBsAg which have the potential to be occupied by the CP-associated antibodies wherein the target epitopes are those defined by the consensus sequence SEQ ID NO:1 on Loop 1 of HBsAg and SEQ ID NO:15 on Loop 2 of HBsAg. Up to four epitopes are assayed or circulating antibodies specific for up to four epitopes provided that at least one epitope is on Loop 1 and one epitope is on Loop 2, as defined by SEQ ID NOs: 1 and 15, respectively. When the clearance profile of epitopes on HBsAg comprise no epitopes available for binding, then the epitopes are deemed “non-available” and this means that the epitopes are occupied by antibodies and a functional cure will occur. Alternatively, if no HBsAg is detected nor any circulating HBV DNA, then a functional cure has been achieved and only the clearance profile of antibodies is present (or can be induced in an immune response following exposure to, and then clearance of HBV).

Accordingly, enabled herein is an assay to determine the likelihood that an individual on treatment for CHB will achieve a functional cure, the assay comprising determining a profile of the availability or non-availability of target epitopes on HBsAg wherein non-availability of the target epitopes is indicative that a functional cure will be achieved, wherein the target epitopes are those defined by the consensus sequence SEQ ID NO:1 on Loop 1 of HBsAg and SEQ ID NO:15 on Loop 2 of HBsAg.

In an embodiment, the assay determines the likelihood that a subject with CHB and on-treatment will achieve a functional cure, the assay comprising determining the presence of a clearance profile of antibodies in the subject which have bound to the clearance epitopes on HBsAg, or which would bind if HBsAg was present, wherein the presence of the clearance profile of antibodies or antibodies complexed to HBsAg is indicative of a functional cure or a likelihood that a functional cure will be achieved, wherein the target epitopes are those defined by the consensus sequence SEQ ID NO: 1 on Loop 1 of HBsAg and SEQ ID NO: 15 on Loop 2 of HBsAg.

Enabled herein is an assay useful for monitoring treatment for CHB, the assay comprising determining a profile of the availability or non-availability of target epitopes on HBsAg wherein non-availability of the target epitopes is indicative that a functional cure will be achieved, wherein the target epitopes are those defined by the consensus sequence SEQ ID NO:1 on Loop 1 of HBsAg and SEQ ID NO:15 on Loop 2 of HBsAg.

As indicated above, up to four epitopes are assayed or circulating antibodies specific for up to four epitopes provided that at least one epitope is on Loop 1 and one epitope is on Loop 2, each as defined by SEQ ID NOs: 1 and 15, respectively.

In an embodiment, described herein is an assay to determine the likelihood that an individual with chronic hepatitis B (CHB) will achieve a functional cure (FC), the assay comprising determining a profile of the availability or non-availability of target epitopes on Hepatitis B virus surface antigen (HBsAg) for binding to anti-HBsAg antibodies wherein the target epitopes are located on Loop 1 ofHBsAg defined by the consensus amino acid sequence:

(SEQ ID NO: 1)
PCX₁TCX₂X₃X₄X₅QGX₆SMX₇PSC

wherein:

### X₁ is K or R;

### X₂ is T or M;

### X₃ is T, or I;

### X₄ is P, T or L;

### X₅ is A or V;

### X₆ is N or T; and

### X₇ is F or Y,

and on Loop 2 of HBsAg defined by the consensus amino acid sequence:

(SEQ ID NO: 15)
CCC X₈KPX₉DGNCTC

wherein:
X₈ is T or S; and;

### X₉ is T or S,

or an amino acid sequence having at least 80% similarity to SEQ ID NO:1 or SEQ ID NO:15 after optimal alignment,
wherein the non-availability of at least one target epitope on each of Loop 1 and Loop 2 for binding to anti-HBsAg antibodies is indicative of those being occupied or blocked by anti-HBsAg antibodies or other molecules from the individual and wherein the individual is deemed likely to achieve a FC whereas availability of the target epitopes for binding to anti-HBsAg antibodies is indicative of the individual not having achieved a FC. Up to four epitopes are assayed or circulating antibodies specific for up to four epitopes provided that at least one epitope is on Loop 1 and one epitope is on Loop 2, each as defined by SEQ ID NOs:1 and 15, respectively.

The term “similarity” as used herein includes exact identity between compared sequences at the amino acid level. Where there is non-identity at the amino acid level, “similarity” includes amino acids that are nevertheless related to each other at the structural, functional, biochemical and/or conformational levels. In an embodiment, amino acid sequence comparisons are made at the level of identity rather than similarity.

Terms used to describe sequence relationships between two or more polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 10 but generally 18 for Loop 1 peptides and 13 for Loop 2 peptides. Because two polypeptides may each comprise (1) a sequence (i.e. only a portion of the complete polypeptide sequence) that is similar between the two polypeptides, and (2) a sequence that is divergent between the two polypeptides, sequence comparisons between two (or more) polypeptides are typically performed by comparing sequences of the two polypeptides over a “comparison window” to identify and compare local regions of sequence similarity. A “comparison window” refers to a conceptual segment of typically 10 contiguous residues that is compared to a reference sequence or 18 for Loop 1 peptides and 13 for Loop 2 peptides. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (1997)Nucl. Acids. Res. 25: 3389. A detailed discussion of sequence analysis can be found in Unit 19.3 of Ausubel et al. (In: Current Protocols in Molecular Biology, John Wiley & Sons Inc. 1994-1998).

The terms “sequence similarity” and “sequence identity” as used herein refers to the extent that sequences are identical or functionally or structurally similar on an amino acid by amino acid basis or over a window of comparison after optimal alignment. Thus, a “percentage of sequence identity”, for example, is calculated by comparing two optimally aligned sequences over the window of comparison, determining the number of positions at which the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage”. Similar comments apply in relation to sequence similarity.

Reference to at least 80% means 80, 81, 82, 83, 84, 85, 86, 87, 88, 89, 90, 91, 92, 93, 94, 95, 96, 97, 98, 99 or 100%.

In another embodiment, disclosed herein is an assay comprising:

(a) immobilizing to a support a set of mAbs, each mAb with a specificity to one of the target epitopes on HBsAg;
(b) contacting the immobilized mAbs with a blood-derived sample from the individual under conditions sufficient to permit capturing of HBsAg if present; and
(c) determining which immobilized mAbs have captured HBsAg and which have not to provide a profile of available and non-available epitopes on HBsAg. As indicated above, up to four epitopes are assayed or circulating antibodies specific for up to four epitopes provided that at least one epitope is on Loop 1 and one epitope is on Loop 2, each as defined by SEQ ID NOs: 1 and 15, respectively.

In an embodiment, monitoring comprises determining the presence of a clearance profile of antibodies in the subject which have bound to the clearance epitopes on HBsAg, or which would bind if HBsAg was present, wherein the presence of the clearance profile of antibodies or antibodies complexed to HBsAg is indicative of a functional cure or a likelihood that a functional cure will be achieved.

Reference to “on-treatment” means the subject is being treated for CHB by the administration of nucleotide or nucleoside analogs (e.g ADV, ETV or TDF), anti-virals, an interferon (e.g. pegIFN-α) or anti-HBV immunoglobulin. The present invention encompasses a human subject which may be referred to as an individual, patient, host, person, target or other like term. However, the protocol of the present invention has been applied to non-human animal models of CHB or its equivalent. Hence, the present protocol has human and animal health applications.

The present assay need not be confined to assessing a subjects' immune response to CHB or treatment of CHB. The protocol can also be effective in determining antibody binding to HBsAg in vitro such as following incubation of antibodies with HBsAg in vitro. Hence, the assay is applicable to determining the availability or non-availability of epitopes on HBsAg following incubation of the antibodies with HBsAg in vitro wherein if an HBsAg-Ab complex forms it can be determined whether the antibodies have bound to the clearance profile of epitopes on the HBsAg. Hence, the subject assay can be used to test a biological sample, culture supernatant, a liquid medium, an excipient medium, a reaction medium and the like. In an embodiment, the biological sample is sera or plasma. The solid discrete units to which test antibody target the clearance profile of epitopes include a collection of magnetic beads, non-magnetic beads and other discrete solid support units to which antibodies or HBsAg-Ab complexes may have bound. The beads can be labeled with a fluorescent label or other suitable label to facilitate flow cytometry.

Enabled herein is an assay to determine the likelihood that an individual with CHB will achieve a functional cure the assay comprising contacting a blood-derived sample comprising a standardized amount of HBsAg from the individual with mAbs specific for HBsAg immobilized to solid discrete unit under conditions sufficient to capture HBsAg complexed with an antibody (anti-HBs) associated with the functional cure of CHB from the individual, wherein the non-detection of captured HBsAg-AB complex indicates the presence of an anti-HBsAg antibody response associated with functional cure, wherein the target epitopes are those defined by the consensus sequence SEQ ID NO:1 on Loop 1 of HBsAg and SEQ ID NO:15 on Loop 2 of HBsAg.

As indicated above, solid discrete units include beads such as magnetic beads. The ability to standardize or normalize the amount of HBsAg added to a test system ensures consistency of results. In addition, for the absence of doubt, up to four epitopes are assayed or circulating antibodies specific for up to four epitopes provided that at least one epitope is on Loop 1 and one epitope is on Loop 2, each as defined by SEQ ID NOs: 1 and 15, respectively.

The specific antibodies are those isolated from an individual who has undergone a functional cure or who subsequently went on to achieve a functional cure during or following anti-HBV treatment. Monoclonal antibodies (mAbs) may also be generated to mimic a patient's antibodies. The anti-HBV treatment may be by any means including by nucleotide or nucleoside analogs anti-viral agents, an interferon and anti-HBV-specific immunoglobulin. It is proposed herein, without limiting the present invention to any one theory or mode of action, that during treatment, a stage arises where the individual's immune system generates an antibody profile which includes a clearance profile of antibodies which leads to a functional cure. In accordance with the present invention, once this has been achieved, treatment can cease or at least be scaled back. Treatment with anti-HBV agents is expensive and requires compliance. The early identification of the development of the clearance profile of antibodies enables treatment to be immediately halted or reduced. This will result in significant economic advantages to patients, to healthcare providers and professionals, healthcare insurers and to the healthcare industry as a whole.

Hence, enabled herein is a method for determining when treatment of an individual for CHB can cease, the method comprising screening for the presence of a profile of antibodies in the individual which constitutes a clearance profile of antibodies which are specific for selected epitopes on HBsAg wherein the presence of the clearance profile of antibodies is indicative of a decision to cease treatment, wherein the target epitopes are those defined by the consensus sequence SEQ ID NO:1 on Loop 1 of HBsAg and SEQ ID NO:15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs: 1 and 15, respectively.

Generally, treatment of CHB is the administration of a nucleotide or nucleoside analog, an anti-viral agent, an interferon or anti-HBV antibodies. The present assay, therefore, has significant economic advantages for the treatment regime for patients with CHB.

Enabled herein is assay to determine the likelihood that an individual with CHB under treatment will achieve a functional cure, the assay comprising contacting a blood-derived sample standardized for level of HBsAg from the individual with mAbs specific for HBsAg immobilized to a solid discrete units under conditions sufficient to capture HBsAg complexed with an antibody (HBsAg-Ab), the antibody associated with the functional cure of CHB from the individual, wherein the non-detection of captured HBsAg-AB complex indicates the presence of an anti-HBsAg antibody response associated with functional cure. In this instance, a clinician may make the decision to cease treatment.

Enabled herein is assay to determine the likelihood that an individual with CHB under treatment will achieve a functional cure the assay comprising contacting a serum sample standardized for level of HBsAg from the individual with mAbs specific for HBsAg immobilized to a solid support via anti-IgG antibodies under conditions sufficient to capture HBsAg complexed with an antibody (HBsAg-Ab) associated with the functional cure of CHB from the individual, wherein the non-detection of captured HBsAg-AB complex indicates the presence of an anti-HBsAg antibody response associated with functional cure, wherein the target epitopes are those defined by the consensus sequence SEQ ID NO: 1 on Loop 1 of HBsAg and SEQ ID NO: 15 on Loop 2 of HBsAg. In this instance, a clinician may make the decision to cease treatment. As indicated above, up to four epitopes are assayed or circulating antibodies specific for up to four epitopes provided that at least one epitope is on Loop 1 and one epitope is on Loop 2, each as defined by SEQ ID NOs: 1 and 15, respectively.

Further, enabled herein is assay to determine the likelihood that an individual with CHB under treatment will achieve a functional cure the assay comprising contacting a serum sample standardized for level of HBsAg from the individual with mAbs specific for HBsAg immobilized to a solid support via anti-IgG antibodies under conditions sufficient to capture HBsAg complexed with an antibody (HBsAg-Ab) associated with the functional cure of CHB from the individual, wherein the detection of captured HBsAg-AB complex at specific epitopes indicates the presence of an anti-HBsAg antibody response not associated with functional cure, wherein the target epitopes are those defined by the consensus sequence SEQ ID NO:1 on Loop 1 of HBsAg and SEQ ID NO:15 on Loop 2 of HBsAg. In this instance, a clinician may make the decision to continue with treatment. Up to four epitopes are assayed or circulating antibodies specific for up to four epitopes provided that at least one epitope is on Loop 1 and one epitope is on Loop 2, each as defined by SEQ ID NOs:1 and 15, respectively.

Taught herein is assay to determine the likelihood that an individual with CHB will achieve a functional cure the assay comprising contacting a blood-derived sample standardized for level of HBsAg from the individual with mAbs specific for HBsAg immobilized to a solid support under conditions sufficient to capture HBsAg complexed with an antibody (HBsAg-Ab) associated with the functional cure of CHB from the individual, wherein the non-detection of captured HBsAg-AB complex at specific epitopes indicates the presence of an anti-HBsAg antibody response associated with functional cure, wherein the target epitopes are those defined by the consensus sequence SEQ ID NO: 1 on Loop 1 of HBsAg and SEQ ID NO: 15 on Loop 2 of HBsAg. As indicated above, up to four epitopes are assayed or circulating antibodies specific for up to four epitopes provided that at least one epitope is on Loop 1 and one epitope is on Loop 2, each as defined by SEQ ID NOs: 1 and 15, respectively.

The term “standardized” also encompasses “normalization” of level of HBsAg or HBsAg-Ab complex and represents optimal level of HBsAg or HBsAg-Ab complex for the assay. A “blood-derived sample” includes serum.

Further enabled herein is an assay to determine the likelihood that an individual with CHB will exhibit a functional cure or has achieved a functional cure, the assay comprising:

(a) immobilizing to one or more solid supports a set of monoclonal antibodies (mAbs), each mAb with a specificity to a select epitope on Hepatitis B virus surface antigen (HBsAg);
(b) contacting the immobilized mAbs with a blood-derived sample putatively comprising HBsAg from the individual under conditions sufficient to permit capturing of HBsAg;
(c) determining which immobilized mAbs have captured HBsAg and which have not to provide a profile of available and non-available epitopes on HBsAg;
wherein a measure of non-availability of select epitopes on HBsAg is indicative of an anti-HBsAg response associated with a functional cure of CHB or a propensity for a functional cure to occur and wherein the target epitopes are those defined by the consensus sequence SEQ ID NO:1 on Loop 1 of HBsAg and SEQ ID NO:15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs:1 and 15, respectively.

Further enabled herein is an assay to determine the likelihood that an individual with CHB will exhibit a functional cure or has achieved a functional cure, the assay comprising:

(a) immobilizing to one or more solid supports a set of monoclonal antibodies (mAbs), each mAb with a specificity to a select epitope on Hepatitis B virus surface antigen (HBsAg);
(b) contacting the immobilized mAbs with a blood-derived sample standardized for a level of HBsAg from the individual under conditions sufficient to permit capturing of HBsAg;
(c) determining which immobilized mAbs have captured HBsAg and which have not to provide a profile of available and non-available epitopes on HBsAg;
wherein a measure of non-availability of select epitopes on HBsAg is indicative of an anti-HBsAg response associated with a functional cure of CHB or a propensity for a functional cure to occur and wherein the target epitopes are those defined by the consensus sequence SEQ ID NO:1 on Loop 1 of HBsAg and SEQ ID NO:15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs:1 and 15, respectively.

Taught herein is an assay to determine the likelihood that an individual with CHB will not exhibit a functional cure, the assay comprising:

(a) immobilizing to one or more solid supports a set of monoclonal antibodies (mAbs), each mAb with a specificity to a select epitope on Hepatitis B virus surface antigen (HBsAg);
(b) contacting the immobilized mAbs with a blood-derived sample standardized for level of HBsAg from the individual under conditions sufficient to permit capturing of HBsAg;
(c) determining which immobilized mAbs have captured HBsAg and which have not to provide a profile of available and non-available epitopes on HBsAg;
wherein a measure of availability of the select epitopes on HBsAg is indicative of an anti-HBsAg response associated with a functional cure of CHB or a propensity for a functional cure to occur and wherein the target epitopes are those defined by the consensus sequence SEQ ID NO: 1 on Loop 1 of HBsAg and SEQ ID NO: 15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs: 1 and 15, respectively.

The present invention is instructional on a method to determine the likelihood that an individual with CHB will exhibit a functional cure or has achieved a functional cure, the assay comprising:

(a) immobilizing to one or more solid supports a set of monoclonal antibodies (mAbs), each mAb with a specificity to a select epitope on Hepatitis B virus surface antigen (HBsAg);
(b) contacting the immobilized mAbs with a blood-derived sample standardized for level of HBsAg from the individual under conditions sufficient to permit capturing of HBsAg;
(c) determining which immobilized mAbs have captured HBsAg and which have not to provide a profile of available and non-available epitopes on HBsAg;
wherein a measure of non-availability of epitopes at Loop 1 and Loop 2 of HBsAg is indicative of an anti-HBsAg response associated with a functional cure of CHB or a propensity for a functional cure to occur and wherein the target epitopes are those defined by the consensus sequence SEQ ID NO:1 on Loop 1 of HBsAg and SEQ ID NO:15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs: 1 and 15, respectively.

Where a functional cure has been achieved or will be achieved based on the assay results, a clinician is then able to decide to cease treatment. Hence, the subject assay can monitor treatment to determine if an individual will achieve a functional cure, determine if an individual has achieved a functional cure and determine if an individual has achieved a cure through natural defence mechanisms.

The clearance epitopes on HBsAg are those that when occupied by antibodies in a subject, the likely result is a functional cure. The epitopes are located on each of Loop 1 and Loop 2 of HBsAg. In relation to the assay, monoclonal antibodies (mAbs) are selected for use in a multiplex assay which target a range of epitopes on HBsAg. One set of mAbs designated mAb5 and 6 targets the Loop 1 epitope and another set of mAb designated mAb7 and 8 targets the Loop 2 epitope selected from:

Loop1 aa 120-137 of HBsAg
SEQ ID NO: 1
P C X₁ T C X₂ X₃ X₄ X₅ Q G X₆ S M X₇ P S C

### X₁ is K or R;

### X₂ is T or M;

### X₃ is T, or I;

### X₄ is P, T or L;

### X₅ is A or V;

### X₆ is N or T; and

### X₇ is F or Y.

Examples includes:

(SEQ ID NO: 2)
P C K T C T T P A Q G N S M F P S C
(SEQ ID NO: 3)
P C R T C T T P A Q G N S M F P S C
(SEQ ID NO: 4)
P C K T C T T P A Q G T S M F P S C
(SEQ ID NO: 5)
P C R T C T T P A Q G T S M F P S C
(SEQ ID NO: 6)
P C K T C T I P A Q G T S M F P S C
(SEQ ID NO: 7)
P C R T C T I P A Q G T S M F P S C
(SEQ ID NO: 8)
P C R T C T I T A Q G T S M F P S C
(SEQ ID NO: 9)
P C R T C T T P A Q G T S M Y P S C
(SEQ ID NO: 10)
P C R T C M T T A Q G T S M Y P S C
(SEQ ID NO: 11)
P C R T C M T T V Q G T S M Y P S C
(SEQ ID NO: 12)
P C R T C T T L A Q G T S M F P S C
(SEQ ID NO: 13)
P C K T C T T L A Q G T S M F P S C;
and
(SEQ ID NO: 14)
P C K T C T T P A Q G N S M Y P S C;
and
Loop2 aa137-149 of HBsAg
SEQ ID NO: 15
C C C X₈ K P X₉ D G N C T C

### X₈ is T or S

### X₉ is T or S.

Examples include:

(SEQ ID NO: 16)
C C C T K P T D G N C T C
(SEQ ID NO: 17)
C C C T K P S D G N C T C
(SEQ ID NO: 18)
C C C S K P T D G N C T C;
and
(SEQ ID NO: 19)
C C C S K P S D G N C T C;

or at least 80% similarity to any one of SEQ ID NOs:1 to 14 or 15 to 19 after optimal alignment. The term “at least 80% similarity” is as defined above. The mAbs 5, 6, 7, and 8 have been previously known and described at RFHBs 1, 2, 4 and 7 by Waters et al. (1986) supra; Waters et al. (1991) supra and Lever et al. (1988) supra.

For the absence of doubt, up to four epitopes are assayed or circulating antibodies specific for up to four epitopes provided that at least one epitope is on Loop 1 and one epitope is on Loop 2, each as defined by SEQ ID NOs:1 and 15, respectively.

In an embodiment the epitopes occupied are those capable of binding or blocking by mAb5 and/or 6 (Loop 1) and mAb7 and/or 8 (Loop 2). The assay may be referred to as a 2-Plex, 3-Plex or 4-Plex assay.

The assay or protocol may be varied without departing from the essence of the present invention. The critical endpoint is the determination of the profile of epitopes on HBsAg which have been occupied by an individual's antibody response. Where the profile of epitopes at Loop 1 and Loop 2 has been occupied, then a functional cure can be expected. Where the antibodies which have the capacity to bind to this profile are present or are indicative upon exposure to HBV but HBsAg is not detectable, then a functional cure has been achieved. At that point, treatment can cease.

Hence, enabled herein is a method of managing treatment of CHB in subjects wherein the treatment comprises the administration of a nucleotide or nucleoside analog, an anti-viral, an interferon or anti-HBs, wherein the method comprises screening a sample from the individual for:

(i) a circulating clearance profile of antibodies which target select epitopes on HBsAg;
(ii) circulating HBsAg which have non-available epitope for binding to this profile of antibodies; or
(iii) circulating HBsAg complexed to antibodies (HBsAg-Ab) which have non-available epitopes for binding to this profile of antibodies, wherein if the profile of antibodies exist in the absence of circulating HBsAg or if HBsAg exist but the epitopes not available for binding, then ceasing the anti-viral treatment, wherein the target epitopes are those defined by the consensus sequence SEQ ID NO: 1 on Loop 1 of HBsAg and SEQ ID NO:15 on Loop 2 of HBsAg.

The present invention encompasses the binding of at least one epitope on Loop 1 (e.g. mAbs5 and 6) and at least one epitope on Loop 2 (e.g. mAbs7 and 8) or a single antibody which blocks the availability of the epitopes on Loop 1 and Loop 2. The individual being assayed may also have the epitopes occupied, not by an immunoglobulin, but by another molecule such as, for example, an innate immunity molecule.

The present invention further contemplates kits for conducting the subject assay. The kit may be in compartment form with one compartment adapted to comprise solid secrete units such as magnetic beads comprising an immobilisation anti-IgG antibody to bind anti-HB sAg epitope antibodies or comprising directly immobilized anti-HBsAg epitope antibodies. A sample is proposed to be added to the first compartment.

The kit may be configured in any number of ways without departing from the essence of the present invention.

### EXAMPLES

Aspects disclosed herein are further described by the following non-limiting Examples.

### Example 1 Development of Clearance Profile Assay

A multiplex bead-based flow cytometric platform is used to develop an HBsAg epitope assay and establish and map the HBsAg profile of epitopes which constitute the clearance profile of HBsAg epitopes (CP HBsAg epitopes). The “clearance profile of epitopes” or “CP-associated epitopes” are those targeted by an individual's antibodies. When all epitopes are bound by the antibodies generated by the individual, clearance of HBsAg can be expected, wherein the target epitopes are those defined by the consensus sequence SEQ ID NO: 1 on Loop 1 of HBsAg and SEQ ID NO: 15 on Loop 2 of HBsAg. Up to four epitopes are assayed or circulating antibodies specific for up to four epitopes provided that at least one epitope is on Loop 1 and one epitope is on Loop 2, each as defined by SEQ ID NOs: 1 and 15, respectively. The assay is derived from an assay by Lim et al. (2014) Hepatology 60 (1) supplement: 1623, 980A. Briefly, the HBsAg multiplex immunoassay comprises panels of fluorescently identified beads each conjugated to a different anti-HBs antibody which binds to HBsAg, followed by a polyclonal phycoerythrin (PE)-conjugated detection antibody. The HBsAg multiplex panels consist of 4 monoclonal antibodies (mAbs) directed against HBsAg ‘a’ determinant and C-terminal domain epitopes spanning residues 99-226 of HBsAg. The HBsAg epitopes are categorized into the following domains: wherein the target epitopes are those defined by the consensus sequence SEQ ID NO:1 on Loop 1 of HBsAg and SEQ ID NO: 15 on Loop 2 of HBsAg. The intensity of PE associated with each bead provides a sensitive measure of antibody recognition of the specific HBsAg epitope within the sample. The epitope profile on HBsAg in comparison to the matched HBV strain backbone has been expressed as fold change in antibody binding or epitope recognition following bioinformatics data analysis. The 95% CI for the normal range of variation of epitope recognition from the reference backbone has been established as +/−0.5 fold change. A gain-of-epitope recognition corresponds to positive fold change (>0.5 fold), and negative fold changes (>−0.5 fold) correspond to a loss or reduction of epitope binding. A depiction of the assay is shown in FIG. 1.

### Example 2 Selection of mAbs

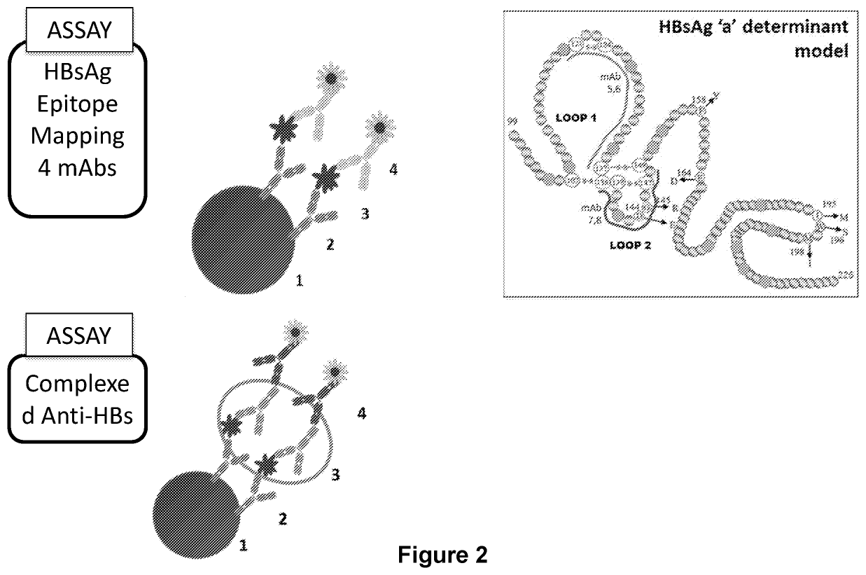

The 4 anti-HBs mAbs utilized in the HBsAg multiplex assay were selected and sourced to provide broad coverage of epitopes with reactivity against the HBsAg Loopl and Loop 2 epitopes wherein the target epitopes are those defined by the consensus sequence SEQ ID NO: 1 on Loop 1 of HBsAg and SEQ ID NO:15 on Loop 2 of HBsAg. The anti-HBs epitope recognition sites are indicated in FIG. 2. The high level of epitope coverage provided by the 4-Plexed mAbs enabled analysis of the HBsAg profile according to the mapped epitope availability, wherein the target epitopes are those defined by the consensus sequence SEQ ID NO:1 on Loop 1 of HBsAg and SEQ ID NO:15 on Loop 2 of HBsAg.

### Example 3 Optimization of Assay/Dynamics

The HBsAg profile assay was developed and optimized using HBV A2 adw2 strain HBsAg, which forms common basis of the majority of generic HBV vaccines and thus represents a relevant baseline or backbone for comparison of HBsAg epitope recognition. The HBsAg source was primarily HBsAg patient sera, confirmed by HBV sequencing as wild-type A2 adw2 (compared to consensus sequences), with confirmation using recombinant wild-type A2 adw2 HBsAg from cell culture supernatant. Assay development was exemplified with mAb5 and/or 6 (Loop 1) and mAb7 and/or 8 (Loop 2). Prepared bead sets that were labeled with a concentration series of the mAbs are incubated against a series of concentrations of HBsAg. These concentrations represent standardization of HBsAg. Conditions were identified which corresponded to comfortable fluorescent readout (within the range of 10000-20000 RFU), without causing overloading and aggregation of the bead/mAb-HBsAg complexes. An assay concentration of HBsAg was determined to be approximately 16 IU/well (the standardized level), with detection of HBsAg as low as 1 IU/well and up to 100 IU/well. A sample dilution series for HBsAg was incorporated in the assay of 8, 16, and 32 IU/well, to account for any slight inaccuracy in the diagnostic determination of HBsAg concentration. The optimal concentration of anti-HBs mAbs labeled to beads was optimized as the mAb concentration resulting in fluorescence reactivity with the dynamic range of the instrument (15000-18000 RFU), and this was specifically/empirically determined for each mAb. The multiplexing of the assay was built by the sequential addition of individual anti-HBs mAbs conjugated to beads, which allowed the identification of epitope competition due to partial shared epitopes by mAbs. To avoid measuring epitope competition between mAbs (i.e. record the HBsAg epitope profile correctly), a 4-Plex anti-HBs bead/mAbs was established, as a 2-Plex, 3-Plex and 4-Plex. HBsAg samples were analyzed in parallel (on the same plate) with these plexes and resulting data combined to achieve a 4-Plex readout of the HBsAg profile.

### Example 4 Assay Validation/HBsAg Profiles Across HBV Strains

The assay was developed against HBsAg A2 adw2, which corresponds to the common vaccine strain of HBV, and thus reflects the HBsAg fingerprint (profile) of epitope recognition for the 4-Plex anti-HBs mAbs in the assay. The assay was validated for both HBsAg A2 adw2 from wild-type ex vivo sera HBsAg and recombinant HBsAg from in vitro cell culture to confirm the HBsAg profile for A2 adw2, which formed a vaccine strain background for the comparison of other HBV strains (genotypes and serotypes), which may be vaccine mismatched, and for reported HBsAg variants with potential for vaccine escape (e.g. sG145R/A, sP120T/L, sD144E/A). The assay was further validated against HBsAg of different genotypes (A-G) and serotypes, sourced from both patient sera and recombinant supernatants, which established the HBsAg profiles of each strain in comparison to the vaccine strain (A2 adw2) background. A reference panel of sera representative of each HBV strain was established for ongoing inclusion in the assay as control data points in study cohorts.

### Example 5 Characterizing Clearance Profile Associated Anti-HBs Response

FIG. 1 shows the application of the assay described in Example 1 to characterizing a clearance profile (CP)-associated with antibodies which result in a functional cure. The assay determines HBsAg epitope occupancy using 4 mAbs wherein mAbs 5 and 6 target the consensus sequence in Loop 1 of:

(SEQ ID NO: 1)
PCX₁TCX₂X₃X₄X₅QGX₆SMX₇PSC

wherein:

### X₁ is K or R;

### X₂ is T or M;

### X₃ is T, or I;

### X₄ is P, T or L;

### X₅ is A or V;

### X₆ is N or T; and

### X₇ is F or Y,

mAbs7 and 8 bind to an epitope within amino acids 137 to 147 of consensus sequence SEQ ID NO:15 in Loop 2 wherein the consensus sequence is:

(SEQ ID NO: 15)
CCC X₈KPX₉DGNCTC

wherein:
X₈ is T or S; and;

### X₉ is T or S.

It is proposed that a population of antibodies which occupy at least one epitope at SEQ ID NO:1 and one epitope at SEQ ID NO:15 represent a clearance profile of antibodies which indicate a functional cure.

Examples of the consensus sequence defined in SEQ ID NO:1 are provided in the amino acid sequences of the polypeptides defined by SEQ ID NOs:2 through 14. Examples of the consensus sequence defined in SEQ ID NO: 15 are provided in the amino acid sequences of the polypeptides defined by SEQ ID NOs: 16 through 19. These peptides include any variants having at least 80% similarity to any one or more of SEQ ID NOs:1 to 14 or 15 to 19 after optimal alignment.

### Example 6 Predictive Biomarkers of Functional CHB Cure

FIG. 2 describes an assay which detects HBsAg with occupied epitopes (Assay 1) and an assay which detects HBsAg complexed to anti-HBs antibodies. The assays employ a 4-Plex immunoassay which detect occupancy of epitopes at Loop 1 (consensus sequence SEQ ID NOs: 1 and 15) and at Loop 2 (consensus sequence SEQ ID NO:15).

### Example 7 HBsAg Clearance Profile in CHB Cure

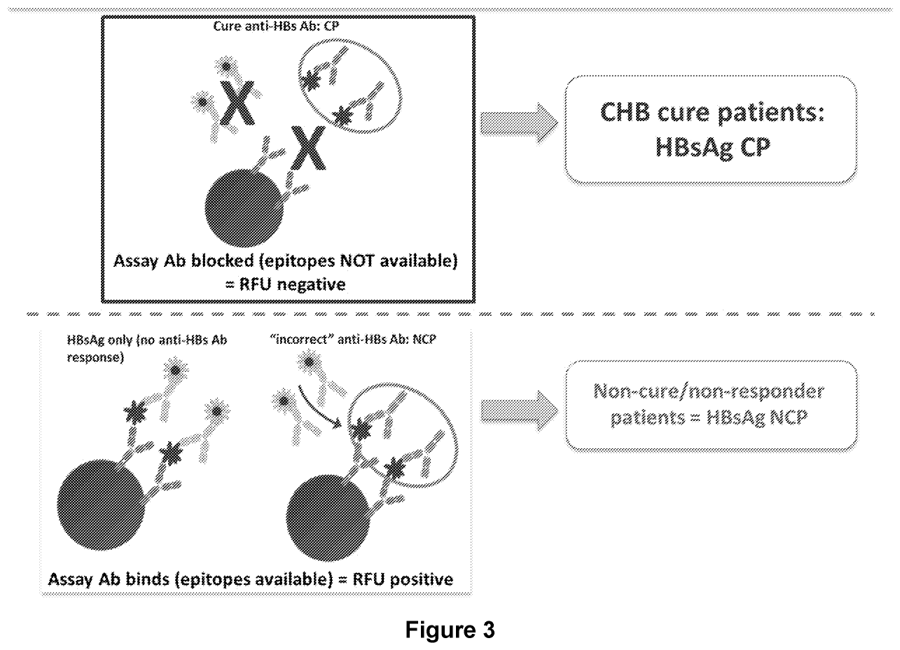

FIG. 3 is a diagrammatic representation showing HBsAg clearance profile (CP) in CHB cure. In FIG. 3A, functional cure patients develop an antibody response that clears infection. In FIG. 3B, non-cure/non-responder patients do not carry the occupied HBsAg epitopes. The term HBsAgCP means the clearance profile of HBsAg epitopes. A HBsAg-non-clearance profile is referred to as HBsAg-NCP.

### Example 8 HBsAg Clearance Profile in CHB Cure

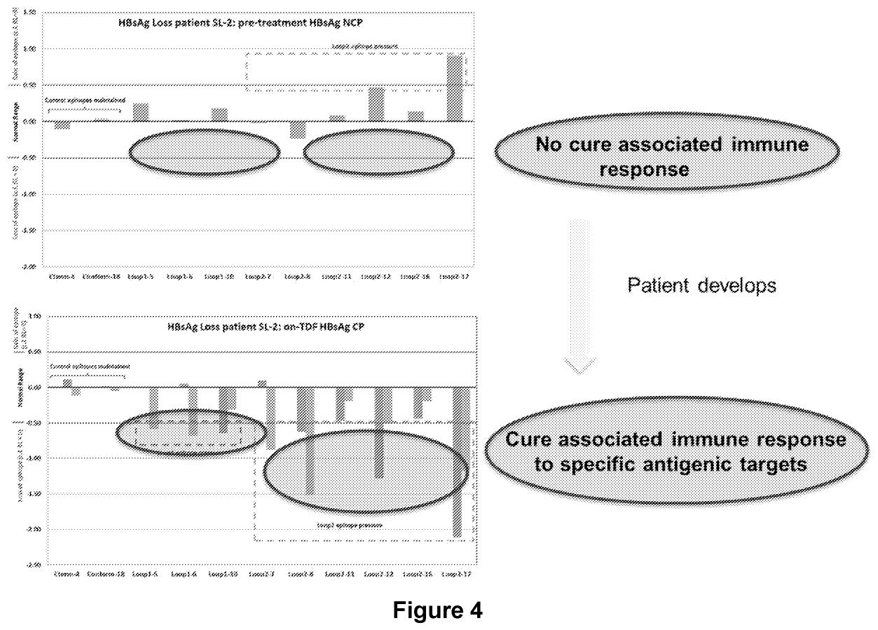

FIG. 4 is a graphical representation showing a patient developing a cure associated immune response which targets specific antigen epitopes.

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

All patents, applications, publications, test methods, literature, and other materials cited herein are hereby incorporated by reference in their entirety as if physically present in this specification.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

Altschul et al. (1997) Nucl. Acids. Res. 25: 3389
Ausubel et al. (In: Current Protocols in Molecular Biology, John Wiley & Sons Inc. 1994-1998
Block et al. (2013) Antiviral Res. 98(1):27-34
Lim et al. (2014) Hepatology 60 (1) supplement: 1623, 980A
Lok et al. (2017) J. Hepatol. 67:847
Volz et al. (2015) International Meeting, Molecular Biology of Hepatitis B virus, Session IX Antiviral therapy, 0-205, Germany
Waters et al. (1986) J. Gen. Hep. 67:2467
Waters et al. (1991) Virus Res. 22:1
Lever et al. (1988) Viral Hep. And Liver Dis.: 961

1. An assay to determine the likelihood that an individual with chronic hepatitis B (CHB) will achieve a functional cure (FC), said assay comprising determining a profile of the availability or non-availability of target epitopes on Hepatitis B virus surface antigen (HBsAg) for binding to anti-HBsAg antibodies wherein the target epitopes are located on Loop 1 of HBsAg defined by the consensus amino acid sequence:
(SEQ ID NO: 1)
PCX₁TCX₂X₃X₄X₅QGX₆SMX₇PSC
wherein:
X₁ is K or R;
X₂ is T or M;
X₃ is T, or I;
X₄ is P, T or L;
X₅ is A or V;
X₆ is N or T; and
X₇ is F or Y,
and on Loop 2 of HBsAg defined by the consensus amino acid sequence:
(SEQ ID NO: 15)
CCC X₈KPX₉DGNCTC
wherein:
X₈ is T or S; and;
X₉ is T or S,
wherein the non-availability of at least one target epitope on each of Loop 1 and Loop 2 for binding to anti-HBsAg antibodies is indicative of those being occupied or blocked by anti-HBsAg antibodies or other molecules from the individual and wherein the individual is deemed likely to achieve a FC whereas availability of the target epitopes for binding to anti-HBsAg antibodies is indicative of the individual not having achieved a FC.

2. The assay of claim 1, wherein said assay comprises contacting a blood-derived sample comprising HBsAg from the individual with monoclonal antibodies (mAbs) specific for the target epitopes on HBsAg, said mAbs immobilized to a solid support, under conditions sufficient to capture HBsAg if the epitopes are not occupied by anti-HBsAg antibodies or other molecules generated by the individual.

3. The assay of claim 1, wherein said assay comprises:
(a) immobilizing to a support a set of mAbs, each mAb with a specificity to one of the target epitopes on HBsAg;
(b) contacting the immobilized mAbs with a blood-derived sample from the individual under conditions sufficient to permit capturing of HBsAg if present; and
(c) determining which immobilized mAbs have captured HBsAg and which have not to provide a profile of available and non-available epitopes on HBsAg.

4. The assay of claim 1, wherein said assay comprises screening for antibodies in the individual which have the capacity to bind to or block the select epitopes on HBsAg wherein the presence of the antibodies in the absence of HBsAg is indicative of the individual having achieved a FC.

5. The assay of any one of claims 1 to 4 wherein the blood-derived sample is serum or a fraction of serum.

6. The assay of claim 5 wherein the serum is standardized for a level of HBsAg.

7. The assay of any one of claims 1 to 4 wherein the individual is under treatment for CHB.

8. The assay of claim 7 wherein the treatment is a nucleotide or nucleoside analog, anti-viral, an interferon or anti-HBV antibodies.

9. The assay of claim 1 wherein non-availability of binding at an epitope defined by SEQ ID NO:1 and SEQ ID NO:15 is indicative of a clearance profile (CP) of antibodies or other molecules associated with a FC and availability of binding an epitope defined by SEQ ID NO:1 and SEQ ID NO:15 is indicative of a non-clearance profile (NCP) of antibodies or other molecules and a FC has not be achieved.

10. The assay of claim 9 wherein the epitope at Loop 1 of HBsAg is defined by an amino acid sequence selected from the group consisting of SEQ ID NOs:2 through 14.

11. The assay of claim 9 wherein the epitope at Loop 2 of HBsAg is defined by an amino acid sequence selected from the group consisting of SEQ ID NOs:16 through 19.

12. The assay of any one of claims 1 to 11 wherein the assay is a 2-Plex, 3-Plex or 4-Plex assay.

13. A method for determining when treatment of an individual for CHB can cease, the method comprising screening for the presence of a profile of antibodies or other molecules in the individual which constitutes antibodies or other molecules associated with a CP which are specific for target epitopes on HBsAg as determined by the assay of any one of claims 1 to 11 wherein the presence of the CP-associated antibodies or other molecules is indicative of a decision to cease treatment and the absence of CP-associated antibodies or other molecules is indicative of a decision not to cease treatment.

14. A method of managing treatment of CHB in an individual wherein the treatment comprises the administration of a nucleotide or nucleoside analog, an anti-viral, an interferon or anti-HBs, wherein the method comprises screening a sample from the individual for:
(i) circulating antibodies or other molecules associated with a CP which the target epitopes on HBsAg;
(ii) circulating HBsAg which have non-available epitopes for binding to this profile of antibodies or other molecules; or
(iii) circulating HBsAg complexed to antibodies (HBsAg-Ab) or other molecules which have non-available epitopes for binding to this profile of antibodies or other molecules;
wherein the target epitopes are determined by the assay of any one of claims 1 to 12, wherein if the profile of antibodies or other molecules exist in the absence of circulating HBsAg or if HBsAg exist but the epitopes not available for binding, then ceasing the anti-viral treatment can be recommended, wherein if the profile of antibodies or other molecules does not exist and circulating HBsAg is present then not ceasing the anti-viral treatment can be recommended.
